(19) United States
(12) Patent Application Publication
(10) Pub. No.: US 2026/0071170 A1
(43) Pub. Date: Mar. 12, 2026

(54) CONTINUOUS ENCAPSULATION OF CELLS IN HYDROGEL TUBES

(71) Applicant: CellGro Technologies, LLC, Lincoln, NE (US)

(72) Inventors: Hendrik J. Viljoen, Ann Arbor, MI (US); Jack T. Rauch, Lincoln, NE (US)

(21) Appl. No.: 19/325,622

(22) Filed: Sep. 11, 2025

Related U.S. Application Data
(60) Provisional application No. 63/693,506, filed on Sep. 11, 2024.

(51) Int. Cl. C12M 1/00; C12M 1/34; C12M 1/36; C12N 5/00

(52) U.S. Cl. CPC C12M 45/22; C12M 23/40; C12M 29/14; C12M 41/44; C12M 41/48; C12N 5/0012

(57) ABSTRACT

Systems and methods for encapsulating cells in hydrogel tubes for cell growth within hydrogel tubes are described. In aspects, a system includes a plurality of peristaltic pumps and a plurality of pulse dampeners to provide oscillatory-controlled flow rates of each of a cell solution, a hydrogel solution, and a cross-linking solution to an extruder configured to form hollow hydrogel tubes having suspended biological cells in an interior.

### CROSS-REFERENCE TO RELATED APPLICATIONS

The present application claims the benefit under 35 U.S.C. § 119 (e) of U.S. Patent Application Ser. No. 63/693,506, filed Sep. 11, 2024, and titled “CONTINUOUS ENCAPSULATION OF CELLS IN HYDROGEL TUBES,” which is herein incorporated by reference in its entirety.

### BACKGROUND

Cell culture involves isolating cells for growth under controlled conditions. Culturing is an important process in biomanufacturing, cell therapies, tissue engineering, regenerative medicine and disease models. Cell therapies and regenerative medicine have high potential in treating many diseases, where cells proliferated by cell culture can provide direct therapies or can be modified for introduction into patients. Cells can be cultured for use as a protein source, where lab-grown food requires large quantities of cultured cells.

### SUMMARY

Systems and methods for encapsulating cells in hydrogel tubes for cell growth within hydrogel tubes are described. In aspects, a system includes a plurality of peristaltic pumps and a plurality of pulse dampeners to provide oscillatory-controlled flow rates of each of a cell solution, a hydrogel solution, and a cross-linking solution to an extruder configured to form hollow hydrogel tubes having suspended biological cells in an interior.

In an aspect, a system includes, but is not limited to, an extruder having a plurality of inlet ports and at least one output port, the plurality of input ports including a first inlet port configured to receive a cell solution containing biological cells, a second inlet port configured to receive a hydrogel solution, and a third inlet port configured to receive a cross-linking solution, a first channel fluidically coupled with the first inlet port to receive the cell solution, a second channel fluidically coupled with the second inlet port to receive the hydrogel solution, the second channel configured to form an annular portion around the first channel, a first chamber configured to permit contact between the cell solution and the hydrogel solution to provide a co-axial fluid stream with the cell solution as a core, a third channel fluidically coupled with the third inlet port to receive the cross-linking solution, and a second chamber fluidically coupled with the third channel and configured to permit contact between the co-axial fluid stream and the cross-linking solution to provide a tri-axial fluid stream forming a hydrogel tube having an interior volume including the cell solution, the interior volume defined by an inner tube diameter, the hydrogel tube having a wall thickness defined between an outer tube diameter and the inner tube diameter; a plurality of peristaltic pumps including a first peristaltic pump configured to introduce the cell solution to the first inlet port, a second peristaltic pump configured to introduce the hydrogel solution to the second inlet port, and a third peristaltic pump configured to introduce the cross-linking solution to the third inlet port; a plurality of pulse dampeners including a first pulse dampener positioned between the first peristaltic pump and the first inlet port, a second pulse dampener positioned between the second peristaltic pump and the second inlet port, and a third pulse dampener positioned between the third peristaltic pump and the third inlet port; a user interface configured to receive a user input of user-specified values for at least one of the inner tube diameter, the outer tube diameter, the inner tube volume, and a flow rate of the cell solution into the extruder; and a system controller communicatively coupled with the user interface and the plurality of peristaltic pumps, the system controller configured to access the user-specified values, initially determine an input flow rate into the extruder for each of the cell solution, the hydrogel solution, and the cross-linking solution based at least in part on the user-specified values, and subsequently determine a dampener flow rate into the plurality of dampeners for each of the cell solution, the hydrogel solution, and the cross-linking solution based on the input flow rate into the extruder for each of the cell solution, the hydrogel solution, and the cross-linking solution.

In an aspect, a system includes, but is not limited to, an extruder having a plurality of inlet ports and at least one output port, the plurality of input ports including a first inlet port configured to receive a cell solution containing biological cells, a second inlet port configured to receive a hydrogel solution, and a third inlet port configured to receive a cross-linking solution, wherein the extruder is configured to permit contact between the cell solution and the hydrogel solution to provide a co-axial fluid stream with the cell solution as a core and to permit contact between the co-axial fluid stream and the cross-linking solution to provide a tri-axial fluid stream forming a hydrogel tube having an interior volume including the cell solution, the interior volume defined by an inner tube diameter, the hydrogel tube having a wall thickness defined between an outer tube diameter and the inner tube diameter; a pump system including a first pump configured to introduce the cell solution to the first inlet port, a second pump configured to introduce the hydrogel solution to the second inlet port, and a third pump configured to introduce the cross-linking solution to the third inlet port; a plurality of pulse dampeners including a first pulse dampener positioned between the first pump and the first inlet port, a second pulse dampener positioned between the second pump and the second inlet port, and a third pulse dampener positioned between the third pump and the third inlet port; a user interface configured to receive a user input of user-specified values for at least one of the inner tube diameter, the outer tube diameter, the inner tube volume, and a flow rate of the cell solution into the extruder; and a system controller communicatively coupled with the user interface and the pump system, the system controller configured to access the user-specified values, determine an input flow rate into the extruder for each of the cell solution, the hydrogel solution, and the cross-linking solution based at least in part on the user-specified values, and determine a dampener flow rate into the plurality of dampeners for each of the cell solution, the hydrogel solution, and the cross-linking solution based on the input flow rate into the extruder for each of the cell solution, the hydrogel solution, and the cross-linking solution.

In an aspect, a method includes, but is not limited to, introducing a flow of a cell solution, via a first peristaltic pump, to a first pulse dampener at a first cell solution flow rate; introducing a flow of a hydrogel solution, via a second peristaltic pump, to a second pulse dampener at a first hydrogel solution flow rate; introducing a flow of a cross-linking solution, via a third peristaltic pump, to a third pulse dampener at a first cross-linking solution flow rate; transferring the cell solution from the first pulse dampener to an extruder at a second cell solution flow rate; simultaneously transferring, with the cell solution from the first pulse dampener, the hydrogel solution from the second pulse dampener to the extruder at a second hydrogel solution flow rate; simultaneously transferring, with the cell solution from the first pulse dampener and with the hydrogel solution from the second pulse dampener, the cross-linking solution from the third pulse dampener to the extruder at a second cross-linking solution flow rate; determining each of the second cell solution flow rate, the second hydrogel solution flow rate, and the second cross-linking solution flow rate based on user-specified values entered into a user interface, the user-specified values including at least one of an inner tube diameter of a hydrogel tube formed by the extruder, an outer tube diameter of the hydrogel tube, an inner tube volume of the hydrogel tube, and a flow rate of the cell solution into the extruder; and determining each of the first cell solution flow rate, the first hydrogel solution flow rate, and the first cross-linking solution flow rate based on each of the second cell solution flow rate, the second hydrogel solution flow rate, the second cross-linking solution flow rate.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### DRAWINGS

The Detailed Description is described with reference to the accompanying figures. In the figures, the use of the same reference numbers in different instances in the description and the figures may indicate similar or identical items.

FIG. 1A is an isometric view of a tri-axial two-port extruder for preparing hydrogel tubes with cell-seeded interior volumes in accordance with example embodiments of the present disclosure.

FIG. 1B is an isometric view of a bi-axial two-port extruder for preparing hydrogel tubes with cell-seeded interior volumes in accordance with example embodiments of the present disclosure.

FIG. 2 is a cross-sectional view of a tri-axial extruder configured to continuously introduce a cell solution into a core of a hydrogel tube formed around the core in accordance with example embodiments of the present disclosure.

FIG. 3 is a schematic illustration of a system for automatically controlling production of hydrogel tubes to provide continuous encapsulation of cells within the tubes in accordance with example embodiments of the present disclosure.

FIG. 4 is a schematic illustration of an implementation of the system of FIG. 3 in accordance with example embodiments of the present disclosure.

FIG. 5 is a cross-sectional view of a pulse dampener of the system of FIG. 3 in accordance with example embodiments of the present disclosure.

FIG. 6 is a chart showing a relationship between logarithmic properties of a pseudo-plastic fluid for the determination of an input flow rate for a pulse dampener in accordance with example embodiments of the present disclosure.

FIG. 7 is a chart showing flow rate over time for an undampened flow from a peristaltic pump and for a dampened flow from the peristaltic pump in accordance with example embodiments of the present disclosure.

FIG. 8A is a schematic illustration of an implementation of the system of FIG. 3 to provide priming of cells with a core solution after pulse dampening in accordance with example embodiments of the present disclosure.

FIG. 8B is a side view of a cell primer of FIG. 8A in accordance with example embodiments of the present disclosure.

FIG. 9A is a microscope picture of a hydrogel tube formed via the system of FIG. 3, with the hydrogel tube shown containing L Wnt-3a cells just after encapsulation.

FIG. 9B is a microscope picture of the hydrogel tube of FIG. 9A, shown containing L Wnt-3a cells three days after encapsulation.

### DETAILED DESCRIPTION

### Overview

Cell culture technology enables production of cells used for treatment of diseases, clinical and laboratory research, disease modeling, drug toxicity studies, and other life-changing uses. For cell growth environments, stirred tank bioreactors can provide environments to culture cells, but such bioreactors do not provide cells with protection from hydrodynamic effects of the internal fluid environment of the bioreactor. Cells are sensitive to their environment and mechanical forces which the surrounding medium exerts on cells reduce growth rates and cause changes in cell properties. Shear stresses in particular have a deleterious effect on the cells, such as causing cells to change their phenotype, causing cell death, and the like. Some suspended cells grow as clusters. When a cluster becomes too big, cells at the center of the cluster are deprived of nutrients and they eventually die. In stirred tank reactors the contents are mixed, which creates shear. The shear can cause cells to detach from a external surface of the cluster, which limits its size, but shear forces damage and/or kill cells.

Micro-environments, such as spaces within bioreactors, can facilitate optimization of cell metabolism while also protecting cells from shear forces in the bioreactor. A hollow hydrogel tube is an example of such a micro-environment, where culturing cells in hydrogel tubes protects cells from mechanical forces present in the bioreactor. After cells have been encapsulated in the hollow tubes, the tubes are placed in a bioreactor together with growth medium. Movement of the growth medium outside the tubes does not lead to shear forces on the cells since the tube wall shields the cells. Encapsulation is the combined process by which hollow hydrogel tubes are fabricated and cells are placed in the hollow space of the hydrogel tubes. The hollow space inside the tube creates ideal conditions for cell growth. There exists a need for large numbers of cells for various industries such as biomanufacturing, cell therapies, tissue engineering, regenerative medicine, and disease models, however the process for producing hydrogel tubes at industrial rates provides many challenges. For instance, automation components, such as fluid pumps, can introduce irregular flow rates or flow pulses, or otherwise provide flow dynamics that can damage hydrogel formation. Further, while syringe pumps can provide regular flow rates, syringe pump volume is limited and unsuited for continuous operations, restricting their use in industrial applications. Still further, syringe pumps, gear pumps, and centrifugal pumps are all in direct contact with the fluids being pumped and generally require a sterilization procedure between subsequent operations, or otherwise are provided as a cost-prohibitive and/or wasteful single-use aspect of the process.

Accordingly, the present disclosure is directed, at least in part, to an automated encapsulation process and system to facilitate substantially continuous formation of hydrogel tubes fabricated to include cells within the hollow space inside the tube. The encapsulation process includes control mechanisms to provide highly consistent and replicable wall thicknesses of the hydrogel tubes to provide a wall thickness that is permeable to cell growth material while maintaining sufficient structural consistency to mitigate irregular flow rates within a bioreactor while facilitating a consistent and continuous product of hydrogel tubes. In aspects, the encapsulation system includes peristaltic pumps to transport fluids, without physical interaction between the fluids and the internal components of the pump, to an extruder that joins fluid flows to form the hydrogel tubes having a cell solution in the core. The extruder is fed with fluid flows including a hydrogel solution, a cell solution, and a cross-linking solution to form the cell-filled hydrogel tubes. By avoiding physical interactions between the pumps and the fluids, the system maintains a sterile environment, such that the pumps do not require a sterilization procedure, thereby increasing uptime on tube production as compared to use of syringe pumps or other pumps that contact the fluid and that process limited volumes of fluid before a pump reset or refill.

However, peristaltic pumps produce flow rate oscillations (e.g., due to rollers pinching fluid lines to move fluid through the lines) that otherwise would cause undesirable deviations in hydrogel tube wall thickness, core size, or combinations thereof, where such deviations can prevent continuous production of substantially uniform hydrogel tubes having a cell solution in the core. The present disclosure mitigates the flow rate oscillations through use of pulse dampeners downstream from the peristaltic pumps, where the pulse dampeners can be tailored to the specific fluid handled by a given peristaltic pump. In aspects, pulse dampeners for the peristaltic pumps that transfer the hydrogel solution and the cell solution have smaller interior volumes as compared to the pulse dampener for the peristaltic pump that transfers the cross-linking solution.

The system includes a computer controller communicatively coupled with the peristaltic pumps to control the flow rates of the fluids through fluid lines of the respective peristaltic pumps. In aspects, the system includes a user interface to receive user input regarding one or more traits (e.g., inner tube diameter, outer tube diameter, inner tube volume, cell solution flow rate, or the like) of the hydrogel tube that are desired as an output of the extruder. The controller can receive the user input and determine the flow rates of the fluids flowing into the extruder and determine what flow rates should be introduced as inputs into the respective peristaltic pumps to generate the determined flow rates of the fluids flowing into the extruder to produce hydrogel tubes having the desired traits in a substantially continuous manner.

### Example Implementations

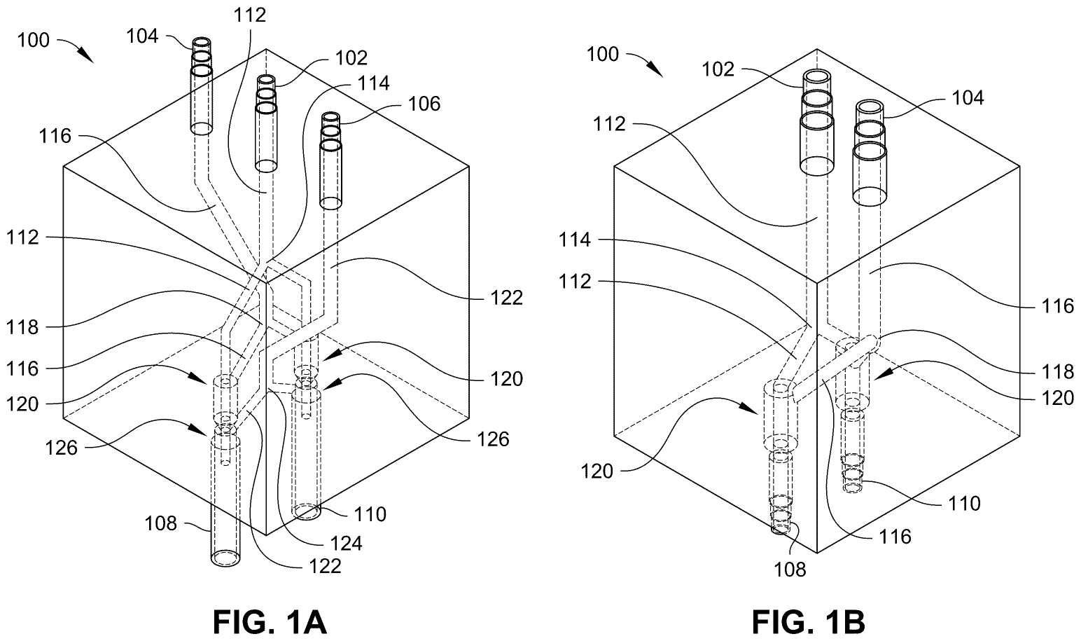

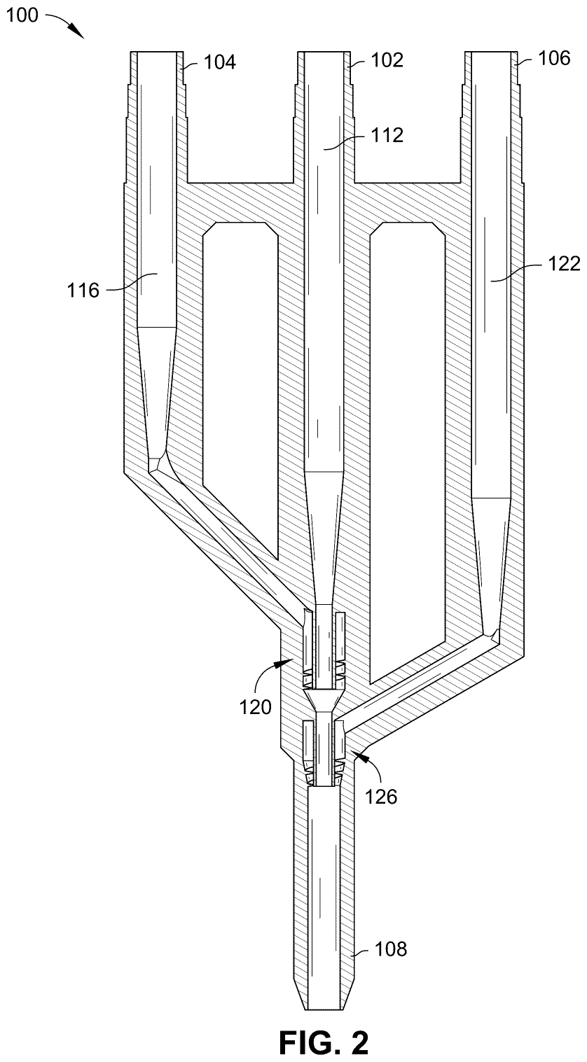

Referring to FIGS. 1A-9B, example implementations of an automated encapsulation process and system to facilitate substantially continuous formation of hydrogel tubes utilizing a multi-axial extruder (“extruder 100”) for producing hydrogel tubes which contain cells within the tube interior are shown, with FIG. 1A illustrating an example of a tri-axial multi-port extruder, FIG. 1B illustrating an example of a bi-axial multi-port extruder, and FIG. 2 illustrating an example of a tri-axial extruder. The extruder 100 is shown including two input ports (ports 102, 104) for a bi-axial extruder and three input ports for a tri-axial extruder (ports 102, 104, 106) and with each extruder 100 shown having one or more output ports (e.g., ports 108, 110). While example extruders 100 are shown with two output ports, the present disclosure is not limited to extruders 100 having two output ports, where extruders 100 having more than two output ports of fewer than two output ports can be provided without departing from the scope of the present disclosure.

The input ports of the extruder 100 are configured to receive fluids to generate fluid-stable hydrogel tubes (e.g., alginate tubes) filled with a core of cell solution for introduction into a cell growth medium, such as by placing the outlet of the extruder 100 above or into a bath containing cell growth medium within, or for transfer to, a bioreactor to proliferate the growth of cells within the hydrogel tubes. For example, the cell solution includes cells for growth suspended in a fluid solution to facilitate transfer through the extruder 100. The cell growth medium can include, but is not limited to, Dulbecco's Modified Essential Medium (DMEM), Fetal Bovine Serum (FBS), Ham's F-12 medium (F12), EA complete medium, and the like, and combinations thereof (e.g., DMEM+10% FBS, DMEM+F12, etc.). For multi-port outlet extruders, the port 102 is coupled with internal channels 112 of the extruder 100 to flow the cell solution through one or more branch portions 114 for distribution of the cell solution from a single input port to multiple outlet ports (e.g., ports 108, 110) in a co-axial arrangement with a hydrogel fluid (e.g., an alginate solution) and, in tri-axial extruders, with a fluid to cross-link the hydrogel (e.g., a calcium chloride solution). Contacting the annulus flow of the hydrogel fluid with calcium chloride causes the hydrogel to polymerize and form a sheath around the core flow of the cell solution. In implementations, a user can identify one or more traits of the sheath (e.g., via a user interface) for a system controller to direct the flow of fluids within the system to automatically form hydrogel tubes having the desired traits.

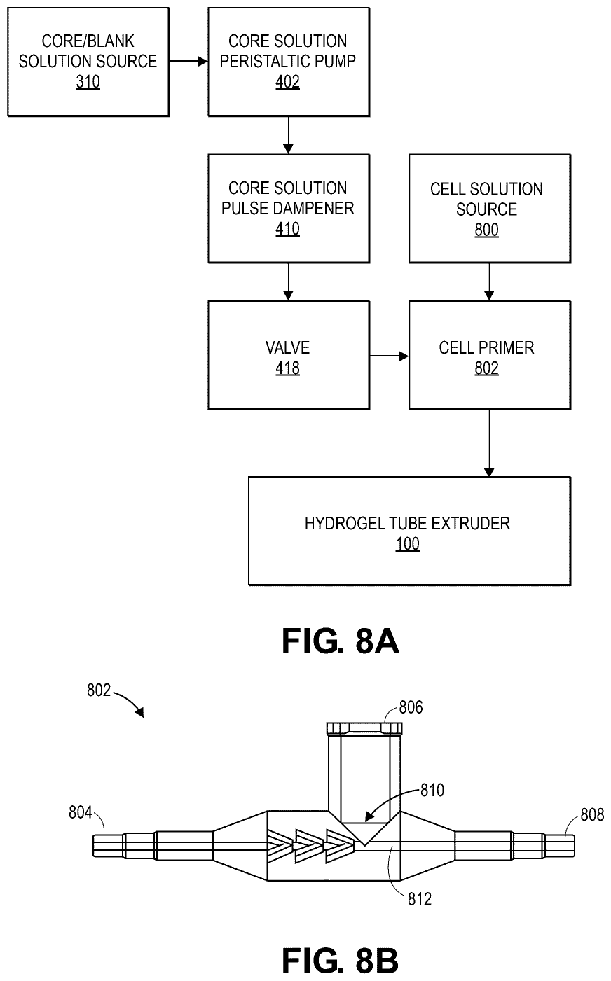

In implementations, the cell solution includes a cell density from about 0.5 million cells/mL to about 10 million cells/mL, however the present disclosure is not limited to such cell densities and can include cells densities less than about 0.5 million cells/mL or cell densities greater than about 10 million cells/mL without departing from the scope of the present disclosure. For cell solutions that are to include substantially more than 10 million cells/mL (e.g., greater than 20 million cells/mL) or that have cells available in relatively small amounts (e.g., less than about 1 million cells/mL), the systems described herein can include a cell priming step between a cell carrier fluid and a cell solution after transfer of the cell carrier fluid through a pulse dampener, as described further herein with respect to FIGS. 8A and 8B).

The port 104 is configured to receive a hydrogel solution (e.g., an alginate solution) and direct the hydrogel solution through internal channels 116 of the extruder 100, and for multi-outlet port extruders, to flow through one or more branch portions 118 for distribution of the hydrogel solution from a single input port to multiple outlet ports (e.g., ports 108, 110), in a co-axial arrangement with the cell solution and, in tri-axial extruders, with the cross-linking solution. For example, the internal channels 116 and the branch portions 118 direct the hydrogel solution to first chambers 120 wherein the hydrogel solution is prepared to flow around the flow of the cell solution in a co-axial arrangement to provide laminar, co-axial flows of the cell solution surrounded by the hydrogel solution. The internal channels 116 and the branch portions 118 are fluidically coupled with the port 104 and are independent of the internal channels 112 and branch portions 114 fluidically coupled with the port 102 to maintain separation of the cell solution and the hydrogel solution until they are flown past each other in the first chambers 120.

For tri-axial extruders 100, the port 106 is configured to receive a cross-linking fluid (e.g., a calcium chloride solution) and direct the cross-linking fluid through internal channels 122 of the extruder 100, and for multi-port outlet extruders to flow through one or more branch portions 124 for distribution of the cross-linking fluid from a single input port to multiple outlet ports (e.g., ports 108, 110), in a co-axial arrangement with the hydrogel solution and the cell solution. For example, the internal channels 122 and the branch portions 124 direct the cross-linking fluid to second chambers 126 wherein the cross-linking fluid is prepared to flow around the flow of the hydrogel fluid in a co-axial arrangement to provide laminar, co-axial flows of the cell solution surrounded by the hydrogel solution (e.g., as an annular portion) and the hydrogel fluid surrounded by the cross-linking fluid. The internal channels 122 and the branch portions 124 are fluidically coupled with the port 106 and are independent of the internal channels 112 and branch portions 114 fluidically coupled with the port 102 and independent of the internal channels 116 and branch portions 118 fluidically coupled with the port 104 to maintain separation of the hydrogel solution with the cell solution core until the cross-linking fluid is flown past the exterior of the hydrogel solution in the second chambers 126. For example, the output from the outlet ports (e.g., ports 108, 110) can include a core of the cell solution surrounded by a layer of hydrogel solution, which in tri-axial extruders 100 is an annulus shape surrounded by a sheath of cross-linking fluid to form hydrogel tubes containing cells within the interior for proliferation of cells in the tube interior within a cell growth medium.

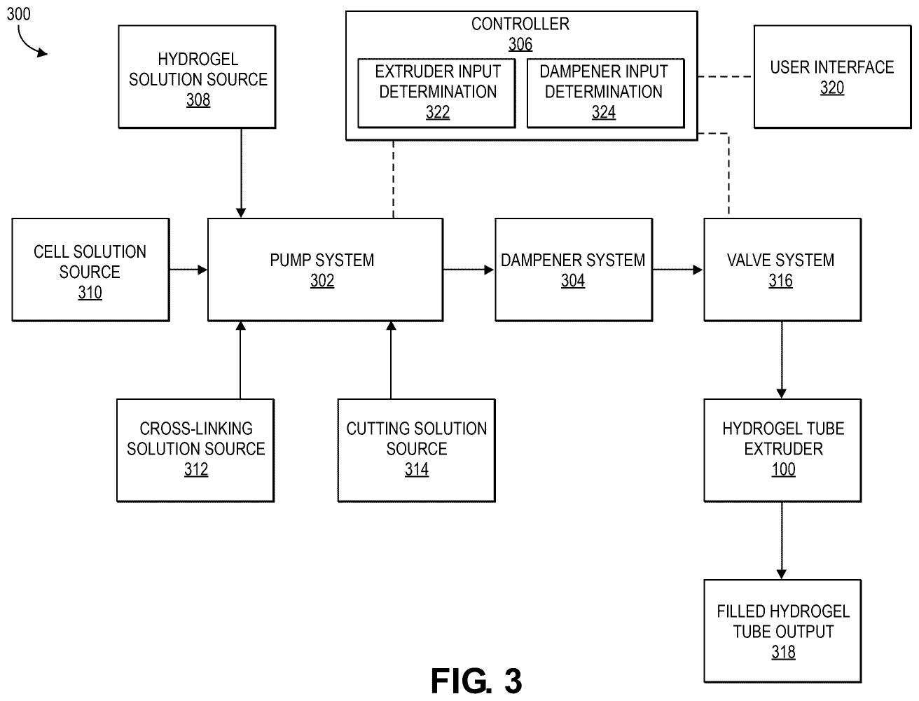

Referring to FIG. 3, a system 300 for automatically producing continuous hydrogel tubes encapsulating of cells within the tubes is shown. The system 300 generally includes a pump system 302, a dampener system 304, the extruder 100, and a controller 306 communicatively coupled with the pump system 302. The pump system 302 is fluidically coupled with fluid sources to fill the extruder 100, where the fluid sources can include a hydrogel solution source 308, a cell solution source 310, a cross-linking solution source 312, and a cutting solution source 314. In general, the pump system 302 operates substantially continuously to introduce the fluid sources to the extruder 100 substantially simultaneously, however, the system 300 is not limited to such operation and can introduce different fluid sources to the extruder 100 at different times, such as to provide periodic introduction of the cell solution from the cell solution source 310 to the extruder 100. The pump system 302 directs the fluids through the dampener system 304 and optionally through a valve system 316 to the inlet ports of the extruder 100 to generate a filled hydrogel tube 318. The valve system 316 can change which fluids are directed to the extruder 100 at a given time, for example, to alternate between directing the cross-linking fluid and the cutting solution to the extruder 100, to mix fluids before directing the mixed fluids to the extruder 100 (e.g., to mix cross-linking solutions, to mix cutting solutions, etc.), or the like.

The controller 306 is communicatively coupled with the pump system 302 to control set points for each pump of the pump system 302 to individually control flow rates of each of the fluids drawn from the hydrogel solution source 308, the cell solution source 310, the cross-linking solution source 312, and the cutting solution source 314. The system 300 is shown including a user interface 320 to receive user input of one or more traits of a desired filled hydrogel tube 318 including, but not limited to, inner tube diameter, outer tube diameter, inner tube volume, and cell solution flow rate into the extruder 100. The controller 306 is communicatively coupled with the user interface 320 to receive the one or more traits to process the traits with respect to an extruder input determination 322 to initially determine what flow rates of each fluid introduced to the extruder 100 would produce filled hydrogel tubes with the user-input traits. Alternatively or additionally, the controller 306 can access a storage device having one or more default values for the traits of the filled hydrogel tube 318, where the controller 306 can determine whether the user provided a sufficient number of user-specified traits or whether one or more default values are to be utilized to initially determine what flow rates of each fluid introduced to the extruder 100 would produce filled hydrogel tubes with the combination of user-input traits and default values. For example, the system 300 can include a default value for a minimum wall thickness and/or a maximum wall thickness such that a user providing an inner tube diameter can establish default values for the outer tube diameter.

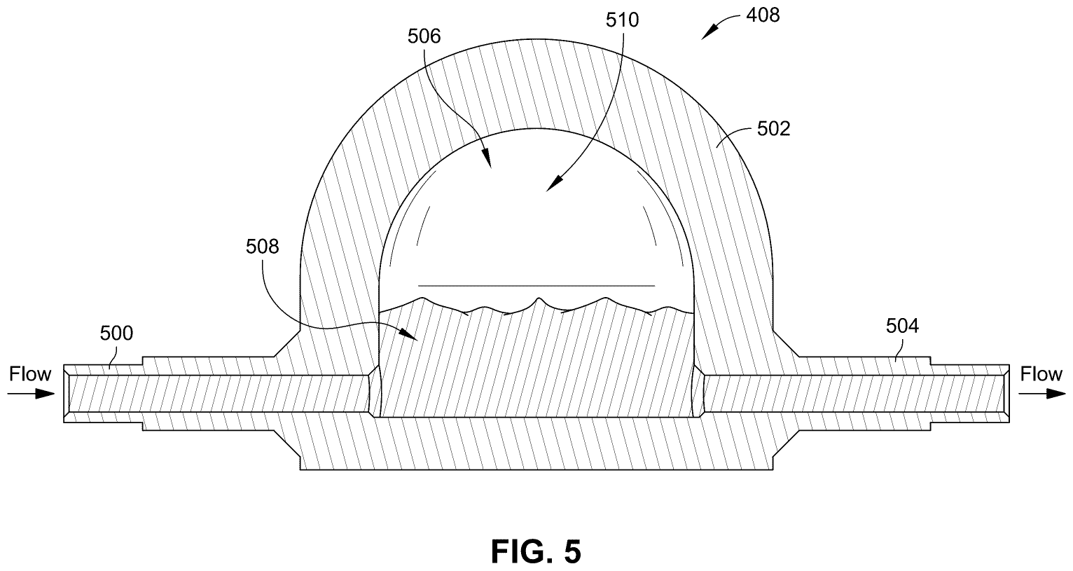

The controller 306 can then utilize a dampener input determination 324 to determine what flow rates of each fluid should be introduced as inputs into the dampener system 304 to generate the determined flow rates from the extruder input determination 322 for introduction to the extruder 100 to produce hydrogel tubes having the desired traits in a substantially continuous manner. For instance, since the dampener system 304 can impact the flow characteristics of flow rates received from the pump system 302, merely controlling the pump system 302 to directly produce the flow rates of each fluid introduced to the extruder 100 could result in undesirable fluctuations in flow rates over time that would cause undesirable deviations in hydrogel tube wall thickness, core size, or combinations thereof, where such deviations can prevent continuous production of substantially uniform hydrogel tubes having a cell solution in the core. For example, for flow rates on the order of microliters per minute (e.g., less than 1 mL/min), even small deviations (e.g., between 100 and 200 μL/min) can result in inconsistent hydrogel tube wall thickness that can negatively impact hydrogel tube length (i.e., discontinuous hydrogel tube production), that are too thick to sufficiently permit diffusion of nutrients into the core of the hydrogel tube, or the like, or combinations thereof. Example flow rate determinations by the controller 306 are described further herein with respect to FIGS. 5 through 7.

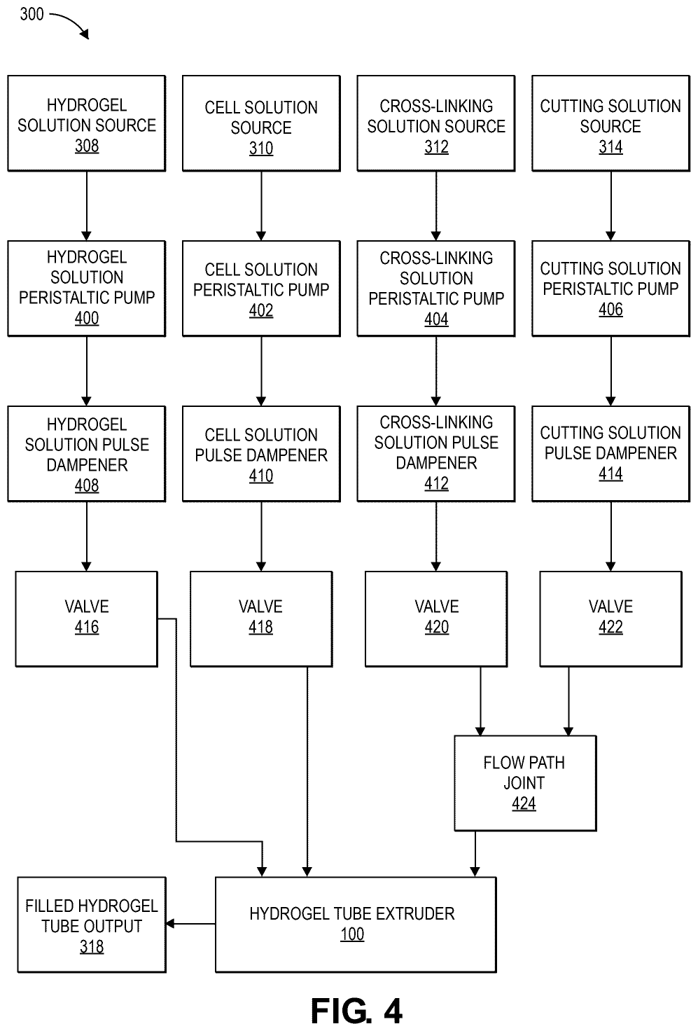

In implementations, the pump system 302 includes a separate pump for each fluid from each of the hydrogel solution source 308, the cell solution source 310, the cross-linking solution source 312, and the cutting solution source 314. For example, FIG. 4 illustrates an embodiment with the pump system 302 including a separate peristaltic pump to draw the fluids from each of the fluid sources. For instance, the system 300 is shown including a hydrogel solution peristaltic pump 400, a cell solution peristaltic pump 402, a cross-linking solution peristaltic pump 404, and a cutting solution peristaltic pump 406. The dampener system 304 can include a separate dampener for each pump of the pump system 304 to dampen the oscillatory effects of the peristaltic pumps on the pump output flow rates, such as due to peristaltic rollers pinching and rolling against fluid lines to draw fluids from each of the hydrogel solution source 308, the cell solution source 310, the cross-linking solution source 312, and the cutting solution source 314 and push the fluids to dampener system 304. For example, the system 300 can include a hydrogel solution pulse dampener 408, a cell solution pulse dampener 410, a cross-linking solution pulse dampener 412, and a cutting solution pulse dampener 414.

The hydrogel solution pulse dampener 408 is fluidically coupled with an outlet of the hydrogel solution peristaltic pump 400 to direct hydrogel solution from the hydrogel solution source 308 to the extruder 100, with an optional valve 416 intervening between the hydrogel solution pulse dampener 408 and the extruder 100. The cell solution pulse dampener 410 is fluidically coupled with an outlet of the cell solution peristaltic pump 402 to direct cell solution from the cell solution source 310 to the extruder 100, with an optional valve 418 intervening between the cell solution pulse dampener 410 and the extruder 100. The cross-linking solution pulse dampener 412 is fluidically coupled with an outlet of the cross-linking solution peristaltic pump 404 to direct cross-linking solution from the cross-linking solution source 312 to the extruder 100, with an optional valve 420 intervening between the cross-linking solution pulse dampener 412 and the extruder 100. The cutting solution pulse dampener 414 is fluidically coupled with an outlet of the cutting solution peristaltic pump 406 to direct cutting solution from the cutting solution source 314 to the extruder 100, with an optional valve 422 intervening between the cutting solution pulse dampener 414 and the extruder 100.

In implementations, the system 300 can include a flow path joint 424 between the outlets of the cross-linking solution pulse dampener 412 and the cutting solution pulse dampener 414. The flow path joint 424 can combine the flows of the cross-linking solution and the cutting solution or can direct one of the flows at a time to the extruder 100 (e.g., through coordination of the valves 420, 422).

### Controller Determination of System Flow Rates

As described herein, the controller 306 can include modules for extruder input determination 322 and dampener input determination 324 based on user-specified traits of the hydrogel tubes. In implementations, the extruder input determination 322 and the dampener input determination 324 utilize fluid dynamics to determine flow rates of each of the hydrogel solution peristaltic pump 400, the cell solution peristaltic pump 402, and the cross-linking solution peristaltic pump 404 to produce substantially continuous hydrogel tubes from the from extruder 100 having the user-specified traits or having traits that are within a threshold deviation from the user-specified traits. An example extruder input determination 322 is provided according to the following.

The radius of the outlet port (e.g., outlet port 108) of the extruder 100 is R₃. The radii of the inlet ports of the extruder 100 to receive the cell solution flow and the hydrogel solution flow (e.g., inlet ports 102, 104, respectively) are R₁ and R₂ respectively. Fluid dynamics principles provide that the axial velocities and shear are continuous at R_{1,2,3}. The cross-linking solution is combined with the co-axial flow, consisting of the core solution and the hydrogel solution, in the extruder port that has a radius R₃. The radial position of the core solution lies between 0 and R₁ and the radial position of the hydrogel solution is between R₁ and R₂. The position of the cross-linking solution is between R₂ and R₃. The cross-linking solution can be construed as a Newtonian fluid with a constitutive relation between the shear stress τ_{rz} and axial velocity gradient

$$\frac{{dv}_{z}}{dr}$$

according to equation (1):

$${τ}_{rz} = \frac{{dv}_{z}}{dr} \qquad ( 1 )$$

where μ is the viscosity of the Newtonian fluid.

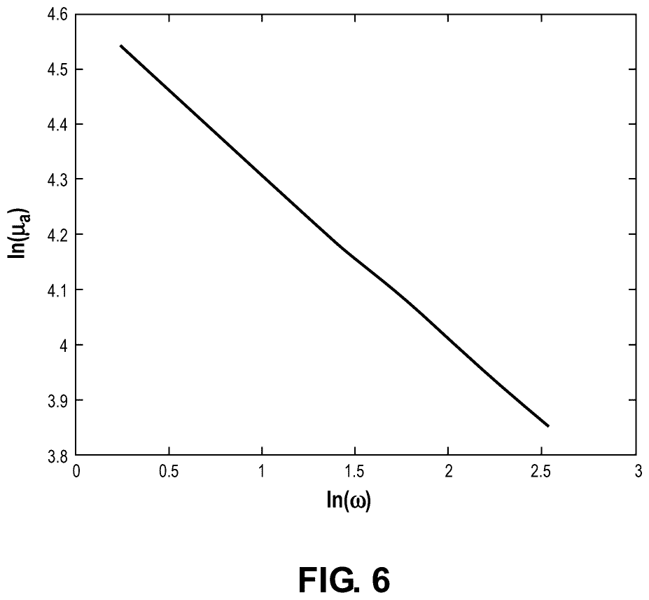

The hydrogel solution and the core solution can be construed as pseudo-plastic fluids, where the respective constitutive relations are provided according to equations (2) and (3):

$${τ}_{rz} = A {( - \frac{{dv}_{z}}{dr} )}^{n} \qquad ( 2 )$$

$${τ}_{rz} = B {( - \frac{{dv}_{z}}{dr} )}^{m} \qquad ( 3 )$$

where A and n are parameters associated with the hydrogel solution, and B and m are parameters associated with the core solution. Examples for the determination of the parameters are described further herein with respect to FIG. 6.

The extruder input determination 322 determines the ratios of the flow rate of the cell solution (Q₁), the flow rate of the hydrogel solution (Q₂), and the flow rate of the cross-linking solution (Q₃) according to equations (4), (5), and (6):

$${Q}_{1} = {π ( β {R}_{3} )}^{2} [ ( \frac{Δ P}{4 μ L} ) {R}_{3}^{2} ( 1 - {α}^{2} ) + {( \frac{Δ P}{2 AL} )}^{1 / n} \frac{n}{n + 1} [ {( α {R}_{3} )}^{( n + 1 ) / n} - {( β {R}_{3} )}^{( n + 1 ) / n} ] ] - π {( \frac{Δ P}{2 BL} )}^{\frac{1}{m}} \frac{2 {{m}^{2} ( β {R}_{3} )}^{\frac{( 3 m + 1 )}{m}}}{( m + 1 ) ( 3 m + 1 )} \qquad ( 4 )$$

$${Q}_{2} = π {R}_{3}^{2} ( {α}^{2} - {β}^{2} ) [ ( \frac{Δ P}{4 μ L} ) {R}_{3}^{2} ( 1 - {α}^{2} ) + {( \frac{Δ P}{2 AL} )}^{1 / n} \frac{n}{n + 1} {( α {R}_{3} )}^{( n + 1 ) / n} ] - {π ( \frac{Δ P}{2 AL} )}^{1 / n} \frac{2 {n}^{2}}{( n + 1 ) ( 3 n + 1 )} [ {( α {R}_{3} )}^{( 3 n + 1 ) / n} - {( β {R}_{3} )}^{( 3 n + 1 ) / n} ] \qquad ( 5 )$$

$${Q}_{3} = ( \frac{Δ P}{8 μ L} ) π {{R}_{3}^{4} ( 1 - {α}^{2} )}^{2} \qquad ( 6 )$$

where L is the length of the outlet port of the extruder, and ΔP is a pressure drop applied across the extruder.

For an example extruder input determination 322, the controller 306 accesses information such that the nozzle radius R₃ of the extruder 100 is 1 mm, with user-specified values of a tube with an inner radius R₁=0.2 mm and outer radius R₂=0.35 mm, which provides that α=0.1225, β=0.04. The cross-linking solution has a viscosity similar to water, such that μ₃=1 mPa·s. The viscosities of the cell solution can be modulated by altering the amount of methyl cellulose added to the cell solution. An equivalent viscosity for pseudo-plastic fluids can be used, as measured by a viscometer at a specific rotation speed. For the example, μ₁=140 mPa·s. The viscosity of the hydrogel solution depends on the concentration of the hydrogel composition (e.g., sodium alginate). For the example, μ₂=550 mPa·s. The controller 306 can then determine that Q₁/Q₃=0.0.0912 and that Q₂/Q₃=0.1881. As such, controller 306 can then determine via the extruder input determination 322 that the ratio of flow rates into the extruder 100 are Q₁:Q₂:Q₃=1:2.06:10.96. In implementations, it has been determined that flow stability of the system 300 is maintained if Q₁<500 microliter/min. As such, the value of Q₁ can set the other two flow rates, where in this example, Q₂=2.0601 and Q₃=10.9601. The extruder input determination 322 can correct the flow rates for swelling as the hydrogel sheath exits the extruder nozzle and the cross-linking fluid cross-links the flow of hydrogel. After the controller 306 determines, via the extruder input determination 322, the flow rates of the cell solution (Q₁), the hydrogel solution (Q₂), and the cross-linking solution (Q₃) going into the extruder 100 to provide the desired hydrogel tube traits, the controller 306 can then implement the dampener input determination 324 to determine the flow rates of the fluids provided by the pumps of the pump system 302 entering the dampener system 304 in order to provide the flow rates determined via the extruder input determination 322, as described further below.

Since peristaltic pumps of the pump system 302 deliver pulsating flows of fluids, the pulsating flows are directed into the dampener system 304 to convert the pulsating flows to quasi-constant flowrates. An example hydrogel solution pulse dampener 408 of the dampener system 304 is shown in FIG. 5, where the hydrogel solution pulse dampener 408 is shown generally including a fluid inlet 500, a dampening chamber 502, and a fluid outlet 504. The dampening chamber 502 receives fluid from the fluid inlet 500 (e.g., via the pump system 302, such as the hydrogel solution peristaltic pump 400) and includes an interior volume (VT) 506 greater than an interior volume of the fluid inlet 500 and the fluid outlet 504 to permit accumulation of fluid within the dampening chamber 502 to allow for pulses of flow from the pump system 302 to be dampened by the volume of accumulated fluid. For instance, the interior volume 506 includes a volume of fluid 508 and a volume of gas 510 (e.g., air, or other ambient or inert gas), where the volume of gas 510 acts against the increase in the volume of fluid 508, but still permits the volume of fluid 508 to change in response to flow rates at the fluid inlet 500 (Qᵢ) and at the fluid outlet 504 (Qₒ). For example, the gas is compressed when the inlet flow increases and expands when the inlet flow decreases. The pressure in the gas pushes the flow out, and although the pressure fluctuates, the output flow is less uneven and the amplitudes of the outgoing oscillating flow are smaller.

The dampener input determination 324 is dependent upon the type of fluid entering the respective pulse dampener. For Newtonian fluids, such as the cross-linking fluid, dampener input determination 324 relates the output flow rate Qₒ to the input flow rate Qᵢ according to equation (7):

$$\frac{{dQ}_{o}}{dt} = \frac{( {Q}_{i} - {Q}_{o} ) {( 2 {P}_{0} {A}^{2} + 16 πμ {LQ}_{o} + ρ {Q}_{o}^{2} )}^{2}}{2 {P}_{0} {V}_{A 0} {A}^{2} ( 16 π μ L + 2 ρ {Q}_{o} )} \qquad ( 7 )$$

where L is the length of the fluid outlet 504 extending from the dampening chamber 502, A is the cross-sectional area of the fluid outlet 504 extending from the dampening chamber 502 perpendicular to the direction of flow, ρ is fluid density, u is the fluid viscosity, and V_{A0} is the air volume 510 in the dampener before the pump is turned on.

For pseudo-plastic fluids, such as the hydrogel solution and the cell solution, the dampener input determination 324 relates the output flow rate Qₒ to the input flow rate Qᵢ according to equation (8):

$$\frac{{dQ}_{o}}{dt} = \frac{( {Q}_{i} - {Q}_{o} ) {( 2 {P}_{0} {A}^{2} + {2}^{2 m + 2} {π}^{\frac{m + 1}{2}} \frac{BL}{{A}^{\frac{3 m + 1}{2}}} {( \frac{3 m + 1}{4 m} )}^{m} {Q}_{o}^{m} + ρ {Q}_{o}^{2} )}^{2}}{2 {P}_{0} {V}_{A 0} {A}^{2} ( {2}^{2 m + 2} {π}^{\frac{m + 1}{2}} \frac{mBL}{{A}^{\frac{3 m + 1}{2}}} {( \frac{3 m + 1}{4 m} )}^{m} {Q}_{o}^{m - 1} + 2 ρ {Q}_{o} )} \qquad ( 8 )$$

where A is the area of the dampener outlet and m and B are based on fluid properties of the fluid, where the m−1 is the slope and In(B) is the intercept of the linear plot of the natural logarithm of ω (the relation between the shear stress and the viscosity gradient for a pseudo-plastic fluid) against the natural logarithm of μₐ (apparent viscosity) as a straight line, using mean squares, according to equation (9):

$$ln ( {μ}_{a} ) = a + b ln ( ω ) \qquad ( 9 )$$

As an example determination of m and B, a viscometer can be used to measure the properties of the pseudo-plastic fluid, where a cylinder is submersed in the pseudo-plastic fluid and the viscometer can rotate the cylinder at different speeds, which can be selected, and the viscosity is measured at each speed. For an example test, the viscosity was measured at 0.2. 0.6, 1.0, 1.5, and 2.0 revolutions per second. The speed is converted to radians per second, ω=2π[0.2; 0.6; 1.0; 1.5; 2.0], where the relationship between the shear stress and the viscosity gradient for a pseudo-plastic fluid is provided according to equation (10):

$${τ}_{r θ} = B {( \frac{\partial {v}_{θ}}{\partial r} )}^{m} \qquad ( 10 )$$

where τ_{rθ} equals the apparent viscosity (μₐ) times the speed in radians per second (ω).

In the example determination, the viscometer measured the apparent viscosities (mPa·s) at the 0.2, 0.6, 1.0, 1.5, and 2.0 revolutions per second speeds as 94, 67, 58, 51, and 47 mPa·s, respectively. The apparent viscosities were shown to decrease with higher speeds, as expected from a pseudo-plastic fluid. The viscosity can therefore be related to K and m according to equation (11):

$${μ}_{a} = {B ( ω )}^{m - 1} \qquad ( 11 )$$

To obtain the pseudo-plastic properties B and m, the natural logarithm of ω is plotted against the natural logarithm of μₐ as a straight line, using mean squares, an example of which is shown in FIG. 6. From the plot shown in FIG. 6, the slope is b=−0.3, the intercept is α=4.6, where the pseudo-plastic parameters are determined by m−1=−0.3; In B=4.6 which result in m=0.7; B=100 mPa·s^{m}. For the determination of n and A (e.g., the parameters associated with the hydrogel solution, the same process for the determination of B and m can be utilized.

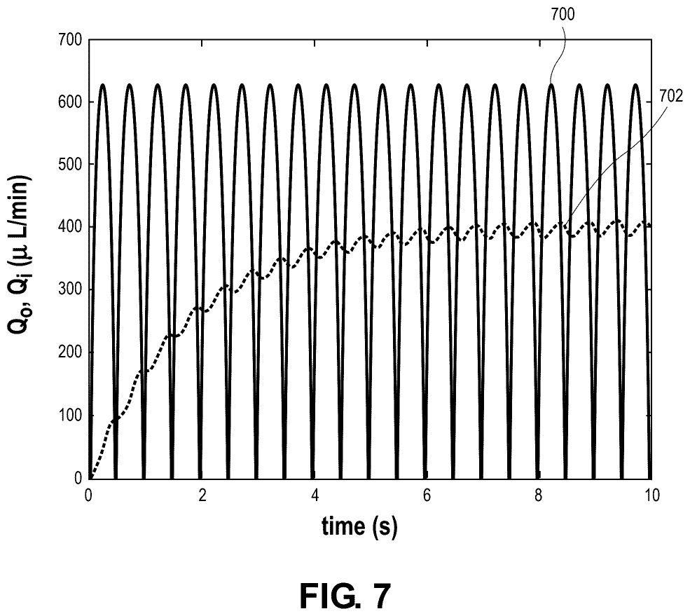

As an example of the extruder input determination 322 and the dampener input determination 324 for the hydrogel solution (e.g., a pseudo-plastic fluid), an alginate solution is to be introduced to the hydrogel solution pulse dampener 408 via the hydrogel solution peristaltic pump, where the alginate solution has the pseudo-plastic properties B and m of (m=0.7, B=100 mPa·s^{0.7}). The user introduces traits of the hydrogel tube via the user interface 320 that cause the controller 306 to determine (e.g., via the extruder input determination 322) a flow rate into the extruder 100 of 400 μL/min for the hydrogel solution. For a dampener volume 506 of 1 cm³ and an initial volume of air 510 of 80%, the flow rate of the hydrogel solution peristaltic pump 400 is determined by the controller 306 as an oscillating flow rate over time (indicated as 700 in FIG. 7) in order to provide the substantially dampened oscillating flow rate over time of the flow rate exiting hydrogel solution pulse dampener 408 (indicated as 702 in FIG. 7). For instance, the undampened flow 700 can be a flow rate from the peristaltic pump of the pump system 302 to a pulse dampener of the dampener system 304, whereas the dampened flow 702 can be a flow rate from the pulse dampener of the dampener system 304 to an inlet of the extruder 100.

The performance of a pulse dampener depends on the viscosity of the fluid. As the viscosity of a fluid increases, it takes a longer time to dampen, but the amplitudes of the oscillations are much reduced. A low viscosity fluid does not dampen easily and a larger volume of air can be utilized to dampen it. Higher viscosity fluids are dampened more than lower viscosity fluids, but they take longer to reach a regular oscillating pattern. If the dampener volume is mostly filled with fluid, the regular oscillating pattern is reached sooner, but if the dampener is mostly filled with gas (like air), then the oscillations are more damped but a longer time is needed to reach the regular pattern. The system 300 herein can facilitate high viscosity fluids (e.g., more than 100 mPa·s) for the cell solution and the hydrogel solution, and low viscosity fluids (e.g., less than 10 mPa·s) for the cross-linking solution. For instance, dampeners having different interior volumes 506 can be utilized to accommodate for the high viscosity and the low viscosity fluids. For example, the hydrogel solution pulse dampener 408 and the cell solution pulse dampener 410 can have smaller interior volumes 506 as compared to the cross-linking solution pulse dampener 412.

### Low Volume of Core Solution/High Cell Count Core Solution

The system 300 can accommodate a variety of cell solutions to be provided as the core within the hydrogel tubes formed by the extruder 100. For example, the system 300 facilitate configurations when a small amount of cells or organoids are available for inclusion in the cell solution source 310 introduced to the cell solution pulse dampener 410 via the cell solution peristaltic pump 402, such as due to the expense or slow growth nature of the cell/organoid line, where the small amount of cells or organoids can be present in an amount less than about 0.5 million cells/mL. As another example, the system 300 can facilitate configurations when a large amount of cells or organoids are available for inclusion in the cell solution source 310 introduced to the cell solution pulse dampener 410 via the cell solution peristaltic pump 402, such as when cells are present in an amount of more than about 20 million cells/mL. For such low and high amounts of cells, the system 300 can introduce the cells as part of a cell solution 800 to a cell primer 802 in combination with a non cell-containing or low cell-containing pulse-dampened flow rate (e.g., a blank solution) received from the core solution pulse dampener 410, for example, as shown in FIGS. 8A and 8B.

The cell primer 802 can include an inlet port 804 to receive the pulse-dampened flow of the blank solution from the core solution pulse dampener 410 and cross-flow inlet port 806 to receive the cell-containing solution from the cell solution 800 to push the cell-containing solution with the blank solution before exiting the cell primer 802 via an outlet port 808 fluidically coupled with the extruder 100 (e.g., via inlet port 102). In implementations, the cross-flow inlet port 806 is perpendicularly arranged with respect to the inlet port 804 and is configured as a luer-lock connection to attach with a syringe loaded with the cell solution 800. The cross-flow inlet port 806 includes a tapered section 810 intersecting with a flow path 812 that extends between the inlet port 804 and the outlet port 808 to introduce the cell solution 800 to the blank solution received through the inlet port 804 while minimizing dead volume within the cell primer 802 with respect to the cell solution 800. For small cell amounts, there is also enough dead volume from the side of the inlet port 804 so that an air gap can be formed between the cell solution 800 and the blank solution to not mix the solutions upstream from the tapered section 810 to have less cell solution that is usable and creates an indicator when the cell solution 800 is close to used up or completely used up.

The system 300 can utilized a priming procedure with the cell primer 802 where the cell primer 802 is initially connected to the syringe filled with the cell solution connected to the cross-flow inlet port 806 and to tubing attached to the outlet port 808 (e.g., coupled with the extruder 100). Then the core line with the blank solution can be primed, filling the line leading out of the core solution pulse dampener 410. After that the tubing from the core line is primed with the blank solution, the tubing can be connected to the inlet port 804 of the cell primer 802. The plunger on the syringe can then be depressed to flow the cell solution 800 through the cell primer 802 and to the empty tubing going to the extruder 100. Since the section of the core line with the blank solution is shut by a valve (e.g., valve 418), the cell solution can only flow to the extruder 100. After introducing the cell solution into the tubing attached to the outlet port 808, the rest of the distance through the extruder 100 can be primed by opening the valve 418, and running the core solution peristaltic pump 402 (e.g., according to control by the controller 306) to have the blank solution flow and the air gap push the cell solution 800 to the extruder 100.

Electromechanical devices (e.g., electrical motors, servos, actuators, or the like) may be coupled with or embedded within the components of the system 300 to facilitate automated operation via control logic embedded within or externally driving the system 300. The electromechanical devices can be configured to cause movement of devices and fluids according to various procedures, such as the procedures described herein. The system 300 may include or be controlled by a computing system having a processor or other controller configured to execute computer readable program instructions (i.e., the control logic) from a non-transitory carrier medium (e.g., storage medium such as a flash drive, hard disk drive, solid-state disk drive, SD card, optical disk, or the like). The computing system can be connected to various components of the system 300, either by direct connection, or through one or more network connections (e.g., local area networking (LAN), wireless area networking (WAN or WLAN), one or more hub connections (e.g., USB hubs), and so forth). For example, the computing system can be communicatively coupled to the pump system 302, the valve system 316, the controller 306, the user interface 320, alternative or additional fluid handling systems (e.g., valves, pumps, etc.), other components described herein, components directing control thereof, or combinations thereof. The program instructions, when executed by the processor or other controller, can cause the computing system to control the system 300 according to one or more modes of operation, as described herein.

It should be recognized that the various functions, control operations, processing blocks, or steps described throughout the present disclosure may be carried out by any combination of hardware, software, or firmware. In some embodiments, various steps or functions are carried out by one or more of the following: electronic circuitry, logic gates, multiplexers, a programmable logic device, an application-specific integrated circuit (ASIC), a controller/microcontroller, or a computing system. A computing system may include, but is not limited to, a personal computing system, a mobile computing device, mainframe computing system, workstation, image computer, parallel processor, or any other device known in the art. In general, the term “computing system” is broadly defined to encompass any device having one or more processors or other controllers, which execute instructions from a carrier medium.

Program instructions implementing functions, control operations, processing blocks, or steps, such as those manifested by embodiments described herein, may be transmitted over or stored on carrier medium. The carrier medium may be a transmission medium, such as, but not limited to, a wire, cable, or wireless transmission link. The carrier medium may also include a non-transitory signal bearing medium or storage medium such as, but not limited to, a read-only memory, a random access memory, a magnetic or optical disk, a solid-state or flash memory device, or a magnetic tape.

### Experimental Operation of the Automated Extruder System

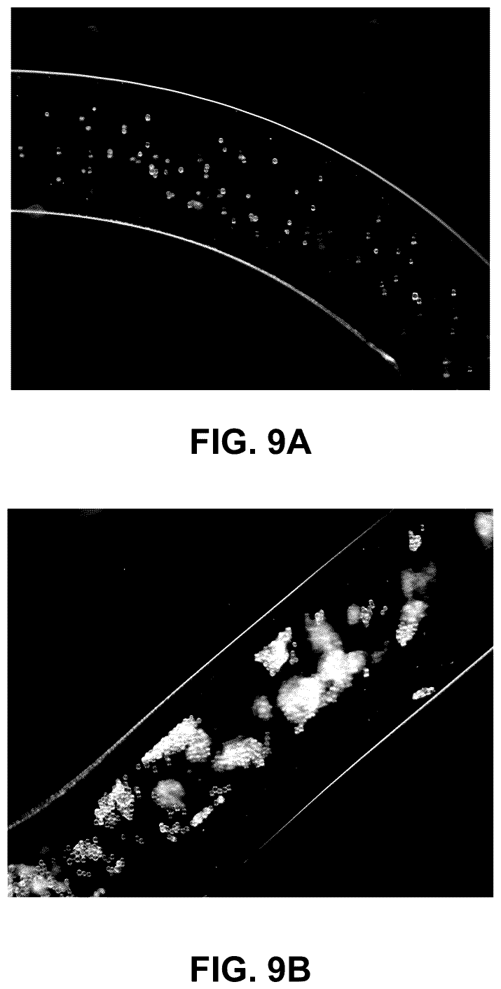

Referring to FIGS. 9A and 9B, example implementations of the system 300 are shown producing hydrogel tubes filled with L Wnt-3a cells deposited into growth medium solutions and subsequent growth of the cells. The controller 306 facilitated control of the pump system 302 to output pump rates of 200 μL/min for the cell solution, 400 μL/min for the hydrogel solution (2% vol. sodium alginate solution)), and 2.5 mL/min for the cross-linking solution (100 mM CaCl₂)). The cell solution was prepared by adding L Wnt-3a cells at 1 million cells/mL. FIG. 9A shows the microscope picture of a tube containing L Wnt-3a cells just after encapsulation. FIG. 9B shows the microscope picture of a tube containing L Wnt-3a cells when three days had passed since encapsulation.

### CONCLUSION

It will be appreciated that features described herein with respect to embodiments or implementations can be combined with any other feature or features described with respect to the same or alternative embodiments, unless context otherwise dictates, without departing from the scope of the present disclosure.

Although the subject matter has been described in language specific to structural features and/or process operations, it is to be understood that the subject matter defined in the appended claims is not necessarily limited to the specific features or acts described above. Rather, the specific features and acts described above are disclosed as example forms of implementing the claims.

1. An encapsulation system for forming hollow hydrogel tubes having suspended biological cells in an interior of the hydrogel tubes, the system comprising:
an extruder having
a plurality of inlet ports and at least one output port, the plurality of input ports including a first inlet port configured to receive a cell solution containing biological cells, a second inlet port configured to receive a hydrogel solution, and a third inlet port configured to receive a cross-linking solution,
a first channel fluidically coupled with the first inlet port to receive the cell solution,
a second channel fluidically coupled with the second inlet port to receive the hydrogel solution, the second channel configured to form an annular portion around the first channel,
a first chamber configured to permit contact between the cell solution and the hydrogel solution to provide a co-axial fluid stream with the cell solution as a core,
a third channel fluidically coupled with the third inlet port to receive the cross-linking solution, and
a second chamber fluidically coupled with the third channel and configured to permit contact between the co-axial fluid stream and the cross-linking solution to provide a tri-axial fluid stream forming a hydrogel tube having an interior volume including the cell solution, the interior volume defined by an inner tube diameter, the hydrogel tube having a wall thickness defined between an outer tube diameter and the inner tube diameter;
a plurality of peristaltic pumps including a first peristaltic pump configured to introduce the cell solution to the first inlet port, a second peristaltic pump configured to introduce the hydrogel solution to the second inlet port, and a third peristaltic pump configured to introduce the cross-linking solution to the third inlet port;
a plurality of pulse dampeners including a first pulse dampener positioned between the first peristaltic pump and the first inlet port, a second pulse dampener positioned between the second peristaltic pump and the second inlet port, and a third pulse dampener positioned between the third peristaltic pump and the third inlet port;
a user interface configured to receive a user input of user-specified values for at least one of the inner tube diameter, the outer tube diameter, the inner tube volume, and a flow rate of the cell solution into the extruder; and
a system controller communicatively coupled with the user interface and the plurality of peristaltic pumps, the system controller configured to access the user-specified values, initially determine an input flow rate into the extruder for each of the cell solution, the hydrogel solution, and the cross-linking solution based at least in part on the user-specified values, and subsequently determine a dampener flow rate into the plurality of dampeners for each of the cell solution, the hydrogel solution, and the cross-linking solution based on the input flow rate into the extruder for each of the cell solution, the hydrogel solution, and the cross-linking solution.

2. The encapsulation system of claim 1, wherein the system controller is configured to control a set point of each of the first peristaltic pump, the second peristaltic pump, and the third peristaltic pump based on the dampener flow rate for each of the cell solution, the hydrogel solution, and the cross-linking solution.

3. The encapsulation system of claim 1, wherein the user interface is configured to receive a user input of user-specified values for at least two of the inner tube diameter, the outer tube diameter, the inner tube volume, and the flow rate of the cell solution into the extruder, and wherein the system controller is configured to determine the input flow rate based at least in part on the user-specified values for at least two of the inner tube diameter, the outer tube diameter, the inner tube volume, and the flow rate of the cell solution into the extruder.

4. The encapsulation system of claim 1, wherein at least one of the first pulse dampener and the second pulse dampener has an interior volume that is smaller than an interior volume of the third pulse dampener.

5. The encapsulation system of claim 1, wherein each of the first pulse dampener and the second pulse dampener has an interior volume that is smaller than an interior volume of the third pulse dampener.

6. The encapsulation system of claim 1, further comprising a valve system fluidically coupled between the plurality of pulse dampeners and the extruder.

7. The encapsulation system of claim 6, further comprising a cell primer positioned between a valve of the valve system and the extruder, the cell primer configured to introduce a bubble between a supply of cells and a blank code solution for transfer of the supply of cells to the extruder.

8. The encapsulation system of claim 7, wherein the cell primer defines a flow path between an inlet port and an outlet port, the inlet port configured to couple with the valve, the outlet port configured to couple with the extruder, and wherein the cell primer defines a cross-flow inlet port configured to couple with the supply of cells to introduce the supply of cells to the flow path.

9. The encapsulation system of claim 8, wherein the cell primer defines a tapered section in the cross-flow inlet port intersecting with the flow path.

10. An encapsulation system for forming hollow hydrogel tubes having suspended biological cells in an interior of the hydrogel tubes, the system comprising:
an extruder having
a plurality of inlet ports and at least one output port, the plurality of input ports including a first inlet port configured to receive a cell solution containing biological cells, a second inlet port configured to receive a hydrogel solution, and a third inlet port configured to receive a cross-linking solution,
wherein the extruder is configured to permit contact between the cell solution and the hydrogel solution to provide a co-axial fluid stream with the cell solution as a core and to permit contact between the co-axial fluid stream and the cross-linking solution to provide a tri-axial fluid stream forming a hydrogel tube having an interior volume including the cell solution, the interior volume defined by an inner tube diameter, the hydrogel tube having a wall thickness defined between an outer tube diameter and the inner tube diameter;
a pump system including a first pump configured to introduce the cell solution to the first inlet port, a second pump configured to introduce the hydrogel solution to the second inlet port, and a third pump configured to introduce the cross-linking solution to the third inlet port;
a plurality of pulse dampeners including a first pulse dampener positioned between the first pump and the first inlet port, a second pulse dampener positioned between the second pump and the second inlet port, and a third pulse dampener positioned between the third pump and the third inlet port;
a user interface configured to receive a user input of user-specified values for at least one of the inner tube diameter, the outer tube diameter, the inner tube volume, and a flow rate of the cell solution into the extruder; and
a system controller communicatively coupled with the user interface and the pump system, the system controller configured to access the user-specified values, determine an input flow rate into the extruder for each of the cell solution, the hydrogel solution, and the cross-linking solution based at least in part on the user-specified values, and determine a dampener flow rate into the plurality of dampeners for each of the cell solution, the hydrogel solution, and the cross-linking solution based on the input flow rate into the extruder for each of the cell solution, the hydrogel solution, and the cross-linking solution.

11. The encapsulation system of claim 10, wherein the system controller is configured to control a set point of each of the first pump, the second pump, and the third pump based on the dampener flow rate for each of the cell solution, the hydrogel solution, and the cross-linking solution.

12. The encapsulation system of claim 10, wherein at least one of the first pulse dampener and the second pulse dampener has an interior volume that is smaller than an interior volume of the third pulse dampener.

13. The encapsulation system of claim 10, further comprising a valve system fluidically coupled between the plurality of pulse dampeners and the extruder.

14. The encapsulation system of claim 13, further comprising a cell primer positioned between a valve of the valve system and the extruder, the cell primer configured to introduce a bubble between a supply of cells and a blank code solution for transfer of the supply of cells to the extruder.

15. The encapsulation system of claim 14, wherein the cell primer defines a flow path between an inlet port and an outlet port, the inlet port configured to couple with the valve, the outlet port configured to couple with the extruder, and wherein the cell primer defines a cross-flow inlet port configured to couple with the supply of cells to introduce the supply of cells to the flow path.

16. The encapsulation system of claim 15, wherein the cell primer defines a tapered section in the cross-flow inlet port intersecting with the flow path.

17. A method for forming hollow hydrogel tubes having suspended biological cells in an interior of the hydrogel tubes, the method comprising:
introducing a flow of a cell solution, via a first peristaltic pump, to a first pulse dampener at a first cell solution flow rate;
introducing a flow of a hydrogel solution, via a second peristaltic pump, to a second pulse dampener at a first hydrogel solution flow rate;
introducing a flow of a cross-linking solution, via a third peristaltic pump, to a third pulse dampener at a first cross-linking solution flow rate;
transferring the cell solution from the first pulse dampener to an extruder at a second cell solution flow rate;
simultaneously transferring, with the cell solution from the first pulse dampener, the hydrogel solution from the second pulse dampener to the extruder at a second hydrogel solution flow rate;
simultaneously transferring, with the cell solution from the first pulse dampener and with the hydrogel solution from the second pulse dampener, the cross-linking solution from the third pulse dampener to the extruder at a second cross-linking solution flow rate;
determining each of the second cell solution flow rate, the second hydrogel solution flow rate, and the second cross-linking solution flow rate based on user-specified values entered into a user interface, the user-specified values including at least one of an inner tube diameter of a hydrogel tube formed by the extruder, an outer tube diameter of the hydrogel tube, an inner tube volume of the hydrogel tube, and a flow rate of the cell solution into the extruder; and
determining each of the first cell solution flow rate, the first hydrogel solution flow rate, and the first cross-linking solution flow rate based on each of the second cell solution flow rate, the second hydrogel solution flow rate, the second cross-linking solution flow rate.

18. The method of claim 17, wherein the extruder comprises:
a plurality of inlet ports and at least one output port, the plurality of input ports including a first inlet port configured to receive the cell solution, a second inlet port configured to receive the hydrogel solution, and a third inlet port configured to receive the cross-linking solution,
a first channel fluidically coupled with the first inlet port to receive the cell solution,
a second channel fluidically coupled with the second inlet port to receive the hydrogel solution, the second channel configured to form an annular portion around the first channel,
a first chamber configured to permit contact between the cell solution and the hydrogel solution to provide a co-axial fluid stream with the cell solution as a core,
a third channel fluidically coupled with the third inlet port to receive the cross-linking solution, and
a second chamber fluidically coupled with the third channel and configured to permit contact between the co-axial fluid stream and the cross-linking solution to provide a tri-axial fluid stream forming the hydrogel tube.

19. The method of claim 17, wherein the extruder includes a plurality of inlet ports and at least one output port, the plurality of input ports including a first inlet port configured to receive the cell solution, a second inlet port configured to receive the hydrogel solution, and a third inlet port configured to receive the cross-linking solution, wherein the extruder is configured to permit contact between the cell solution and the hydrogel solution to provide a co-axial fluid stream with the cell solution as a core and to permit contact between the co-axial fluid stream and the cross-linking solution to provide a tri-axial fluid stream forming the hydrogel tube.

20. The method of claim 17, wherein determining each of the first cell solution flow rate, the first hydrogel solution flow rate, and the first cross-linking solution flow rate based on each of the second cell solution flow rate, the second hydrogel solution flow rate, the second cross-linking solution flow rate comprises:
determining of the first cell solution flow rate based on the second cell solution flow rate and an interior volume of the first pulse dampener,
determining the first hydrogel solution flow rate based on the second hydrogel solution flow rate and an interior volume of the second pulse dampener, and
determining the first cross-linking solution flow rate based on the second cross-linking solution flow rate and an interior volume of the third pulse dampener.
